(19) United States
(12) Plant Patent Application Publication
(10) Pub. No.: US 2017/0020045 P1
(43) Pub. Date: Jan. 19, 2017

(54) Strawberry plant named 'DRISSTRAWFORTYNINE'

(50) ;

(71) Applicant: DRISCOLL STRAWBERRY ASSOCIATES, INC., Watsonville, CA (US)

(72) Inventors: Esther Kibbe, Tampa, FL (US); Philip J. Stewart, Watsonville, CA (US); Arcelia C. Mojica, Plant City, FL (US)

(21) Appl. No.: 14/545,991

(22) Filed: Jul. 15, 2015

(51) Int. Cl. A01H 5/00

(52) U.S. Cl. PLT/208

(57) ABSTRACT

A new and distinct variety of strawberry plant named ‘DrisStrawFortyNine’ particularly characterized by having medium-sized, medium red fruit, a flat globose plant habit, and early harvest maturity, is disclosed.

### GENUS AND SPECIES

Fragaria×ananassa

### VARIETY DENOMINATION

‘DrisStrawFortyNine’

### BACKGROUND OF THE NEW PLANT

The present invention relates to a new and distinct strawberry variety designated ‘DrisStrawFortyNine’ and botanically known as Fragaria×ananassa. This new strawberry variety was discovered in Hillsborough County, Fla. in December 2009 and originated from a cross between the proprietary female parent ‘18Q361’ (unpatented) and the proprietary male parent ‘68N66’ (unpatented). A single plant was selected and asexually propagated via tissue culture and vegetative cuttings in Shasta County, Calif. in 2009.

‘DrisStrawFortyNine’ underwent further testing in Hillsborough County, Fla. from 2009-2014. The present invention has been found to retain its distinctive characteristics through successive asexual propagations via stolons and tissue culture.

### SUMMARY OF THE INVENTION

The following are the most outstanding and distinguishing characteristics of this new cultivar when grown under normal horticultural practices in Hillsborough County, Fla.
1. Medium-sized, medium red fruit;
2. Flat globose plant habit; and
3. Early harvest maturity.

### DESCRIPTION OF THE PHOTOGRAPHS

The accompanying color photographs show typical specimens of the new variety at various stages of development. The colors shown are as true as can be reasonably obtained by conventional photographic procedures. The photographs were taken from three-month-old plants.

FIG. 1 shows upper and lower surfaces of the leaves of the plant with three leaflets.

FIG. 2 shows both upper and lower surfaces of the flowers.

FIG. 3 shows the whole fruit.

FIG. 4 shows the fruit in longitudinal cross-section.

FIG. 5 shows the entire plant habit.

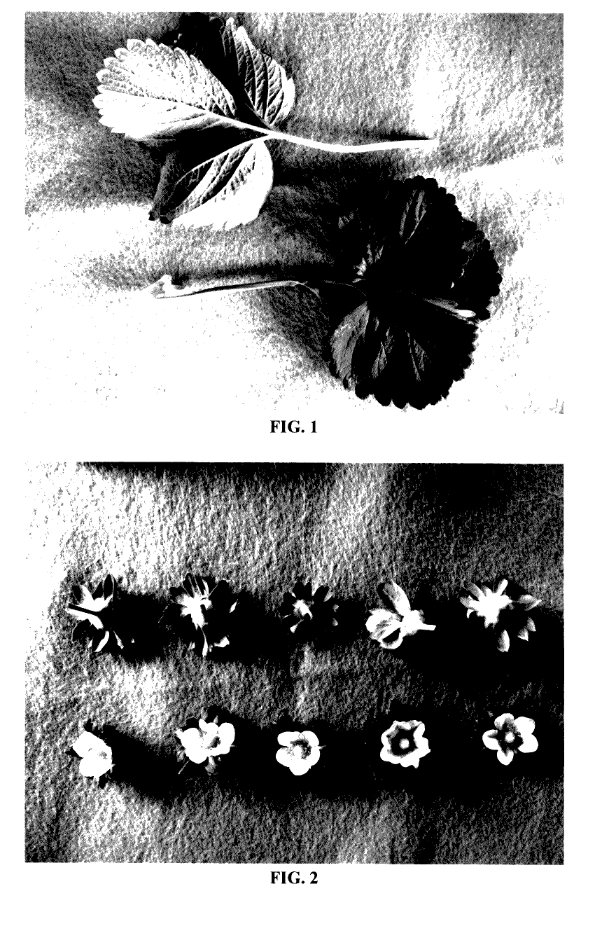

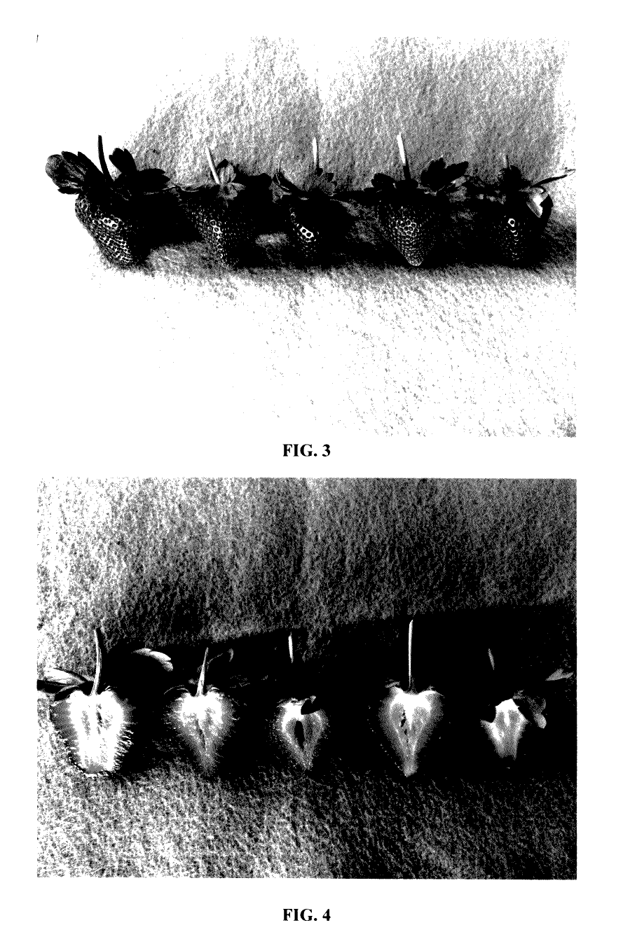

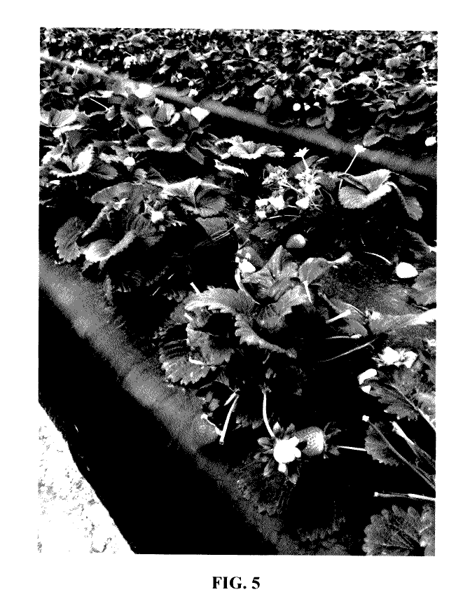

### DESCRIPTION OF THE NEW VARIETY

The following detailed descriptions set forth the distinctive characteristics of ‘DrisStrawFortyNine’. The data which define these characteristics is based on observations taken in Hillsborough County, Fla. from 2009 to 2014. This description is in accordance with UPOV terminology. Color designations, color descriptions, and other phenotypical descriptions may deviate from the stated values and descriptions depending upon variation in environmental, seasonal, climatic, and cultural conditions. ‘DrisStrawFortyNine’ has not been observed under all possible environmental conditions. The botanical description of ‘DrisStrawFortyNine’ was taken from three-month-old plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.

### DETAILED BOTANICAL DESCRIPTION OF THE PLANT

Classification:
   Species.—Fragaria×ananassa.
   Common name.—Strawberry.
   Denomination.—‘DrisStrawFortyNine’.
Parentage:
   Female parent.—The proprietary variety ‘18Q361’ (unpatented).
   Male parent.—The proprietary variety ‘68N66’ (unpatented).
Plant:
   Height.—10.6 cm.
   Diameter.—29.8 cm.
   Number of crowns/plant.—3.
   Habit.—Flat globose.
   Density of individual plant.—Open — sparse.
   Vigor (health and hardiness of plant).—Weak to medium.
Terminal Leaflets:
   Size.—Medium. Length: 7.7 cm Width: 8.4 cm Length/width ratio: 0.9 (Broader than long).
   Number of teeth/terminal leaflet.—25.
   Shape of teeth.—Obtuse — serrate to crenate.
   Color.—Upper surface: RHS 137A (Medium green) Lower surface: RHS 143D (Light green).
   Shape in cross section.—Slightly concave.
   Blistering.—Weak.
   Glossiness.—Absent or weak.
   Number of leaflets.—Three only.
   Shape.—Orbicular.
   Base shape.—Rounded.
   Apex descriptor.—Rounded.
   Margin.—Crenate.
   Margin profile.—Revolute (margins rolled backwards).
   Variegation.—Absent.
Petiole:
   Length.—Short; 10.5 cm.
   Diameter.—2.45 mm.
   Pubescence.—Dense.
   Pose of hairs.—Outwards — horizontal.
   Color.—RHS 141D (Green).
   Bract frequency.—0.
Petiolule:
   Length.—6.16 mm.
   Diameter.—1.26 mm.
   Color.—RHS 141D (Green).
Stipule:
   Length.—3.1 cm.
   Width.—8.73 mm.
   Pubescence.—Sparse.
   Stipule anthocyanin coloration.—Absent or very weak; RHS 145C (Yellow-green).
Stolon:
   Number.—Medium.
   Average number of daughter plants per square foot.—13.
   Anthocyanin coloration.—Strong; RHS 53D (Red).
   Diameter at bract.—1.08 mm.
   Density of pubescence.—Absent or very sparse.
Inflorescence:
   Position relative to foliage.—Above.
   Number of flowers per plant.—Medium; 2.50.
   Time of flowering (50% of plants at first flower).—Early; Mid-November to late March.
   Flower size.—Medium.
   Flower diameter.—25.74 mm.
   Petals.—Shape: Orbicular Apex: Rounded Base: Concavo-convex Margin: Entire Spacing: Overlapping Length: 10.82 mm Width: 11.23 mm Length/width ratio: 1.0 (As long as broad) Petal number per flower: 6 Color (upper surface): RHS NN155B (White).
   Calyx.—Diameter: 50.86 mm Diameter relative to corolla: Much larger Inner calyx diameter relative to outer: Smaller Insertion of calyx: In a basin — inserted Pose of calyx segments: Spreading — outwards Size of calyx in relation to fruit: Slightly larger Adherence of calyx: Strong.
   Sepal.—Shape: Oblong Apex: Convex Margin: Entire Length: 19.44 mm Width: 11.41 mm Sepal number: 13.
   Receptacle color.—RHS 149B (Yellow-green).
   Stamen.—Present Anther color: RHS 13A (Yellow).
   Pedicel.—Attitude of hairs: Upwards.
Fruiting truss:
   Length.—Short; 18.2 cm.
   Diameter at base of truss.—3.38 mm.
   Number of berries per fruiting truss.—2.
   Attitude at first picking.—Prostrate.
   Color at base of truss.—RHS 143C (Green).
Fruit:
   Relative fruit size.—Medium.
   Length.—46.49 mm.
   Width.—40.20 mm.
   Length/width ratio.—1.2 (Longer than broad).
   Fruit hollow length.—13.45 mm.
   Fruit hollow width.—3.54 mm.
   Fruit hollow length/width ratio.—3.8 (Longer than broad).
   Fruit hollow center (cavity).—Small.
   Fruit weight.—27.0 g.
   Predominant fruit shape.—Conical.
   Difference in shape between primary and secondary fruits.—Slight.
   Evenness of fruit surface.—Even or very slightly uneven.
   Fruit skin color.—RHS 45B (Medium red).
   Evenness of fruit color.—Even or very slightly uneven.
   Fruit glossiness.—Medium.
   Achenes.—Insertion of achenes: Level with surface Coloration (sunward side of berry): RHS 59C (Red-purple) Coloration (shaded side of berry): RHS 151B (Yellow-green) Number per berry: 378 Weight (weight of achenes divided by total # seed): 0.000528 g Width of band without achenes: Medium.
   Firmness of flesh (when fully ripe).—Firm.
   Color of flesh (excluding core).—RHS 42B (Dark red).
   Color of core.—RHS 39A (Medium red).
   Evenness of flesh color.—Slightly uneven.
   Distribution of flesh color.—Marginal and central.
   Sweetness.—Medium.
   Acidity.—Medium.
   Texture when tasted.—Fine.
   Type of bearing.—Partially everbearing — partially remontant.
   Harvest interval.—Early December to late March.
   Harvest maturity.—Early.
   Production.—385.28 grams per plant.
Disease and pest resistance:
   Tetranychus urticae.—Susceptible.
   Botrytis fruit rot.—Susceptible.
   Xanthomonas fragariae.—Moderately susceptible.
   Colletotrichum gloeosporioides.—Susceptible.
Stress resistance:
   High temperatures.—Moderately resistant.
   Wind.—Moderately susceptible.
   High pH.—Resistant.

### COMPARISON WITH PARENTAL AND COMMERCIAL VARIETIES

When ‘DrisStrawFortyNine’ is compared to the female parent ‘18Q361’ (unpatented), ‘DrisStrawFortyNine’ is later to come into production and has less yield per plant than ‘18Q361’. Additionally, ‘DrisStrawFortyNine’ plants are larger and healthier than those of ‘18Q361’.

When ‘DrisStrawFortyNine’ is compared to the male parent ‘68N66’ (unpatented), ‘DrisStrawFortyNine’ produces earlier and has higher overall season yields than ‘68N66’. Additionally, ‘DrisStrawFortyNine’ has smaller fruit size than ‘68N66’.

‘DrisStrawFortyNine’ differs from the commercial variety ‘DrisStrawTwentyFour’ (U.S. Plant Pat. No. PP23,378), in that ‘DrisStrawFortyNine’ has medium-sized fruit that is medium red color with medium glossiness, whereas ‘DrisStrawTwentyFour’ has very large fruit that is dark red color with strong glossiness. Additionally, ‘DrisStrawFortyNine’ has inflorescence positioned above the foliage, whereas ‘DrisStrawTwentyFour’ has inflorescence positioned beneath the foliage.

‘DrisStrawFortyNine’ differs from the commercial variety ‘DrisStrawTwelve’ (U.S. Plant Pat. No. PP21,538), in that ‘DrisStrawFortyNine’ has an open-sparse plant density and inflorescence positioned above the foliage, whereas ‘DrisStrawTwelve’ has a medium plant density and inflorescence that is positioned beneath the foliage. Additionally, ‘DrisStrawFortyNine’ has medium-sized fruit with medium red color, whereas ‘DrisStrawTwelve’ has large fruit with dark purple-red color.

We claim:

1. A new and distinct variety of strawberry plant named ‘DrisStrawFortyNine’, substantially as illustrated and described herein.
